(19) United States
(12) Patent Application Publication
(10) Pub. No.: US 2021/0398624 A1
(43) Pub. Date: Dec. 23, 2021

(54) SYSTEMS AND METHODS FOR AUTOMATED INTAKE OF PATIENT DATA

(71) Applicant: Harrow IP, LLC, Nashville, TN (US)

(72) Inventors: Mark L. BAUM, Nashville, TN (US); Andrew E. LIVINGSTON, Nashville, TN (US)

(21) Appl. No.: 17/355,001

(22) Filed: Jun. 22, 2021

Related U.S. Application Data
(60) Provisional application No. 63/042,465, filed on Jun. 22, 2020.

(51) Int. Cl. G16H 10/60; G16H 40/20; G06F 40/40; G06F 16/25; G06N 3/02

(52) U.S. Cl. CPC G16H 10/60; G16H 40/20; G06N 3/02; G06F 16/258; G06F 40/40

(57) ABSTRACT

Some embodiments of the present disclosure disclose methods and systems for automated intake of patient data, and more specifically, for generating structured database of patients and real-time identification of health care providers for the patients based on the received real-time patient data. A patient intake server may receive from a patient intake device, a patient intake data including unstructured patient intake data related to personal or medical information of the patient. The patient intake server may select an engine configured to process the unstructured patient intake data to convert the unstructured patient intake data into a structured patient intake data. In some embodiments, the patient intake server may identify one or more health care providers qualified to provide the medical care to the patient based on an analysis of the structured patient intake data.

### CROSS-REFERENCE TO RELATED APPLICATIONS

The present application claims priority to and the benefit of the U.S. Provisional Patent Application No. 63/042,465, filed Jun. 22, 2020, titled “Systems and Methods for Automated Intake of Patient Data,” which is hereby incorporated by reference in its entirety as if fully set forth below and for all applicable purposes.

### TECHNICAL FIELD

The present disclosure relates generally to systems and methods for automated intake of patient data, and more specifically, for generating structured database(s) of patients and real-time identification of health care providers for the patients based on the received patient intake data.

### BACKGROUND

Patients in search of medical care at health care facilities are usually requested to provide personal and medical data upon arrival. In most cases, the patient data intake process is paper-based and as such can be time consuming, labor intensive and repetitive. Further, patients usually do not receive assistance when providing the patient data, resulting in patient frustration and inaccuracies in the provided data.

### BRIEF DESCRIPTION OF THE DRAWINGS

FIG. 1 is a simplified diagram of a system for automated intake of patient data, according to some embodiments of the present disclosure.

FIG. 2 is a simplified flowchart of a method for automated intake of patient data, according to some embodiments of the present disclosure.

FIG. 3 is an example block diagram illustrating a computer system for implementing a method for automated intake of patient data, according to some embodiments of the present disclosure.

FIG. 4 illustrates an example neural network that can be used to implement a computer-based model according to various embodiments of the present disclosure.

In the figures and appendix, elements having the same designations have the same or similar functions.

### BRIEF SUMMARY OF SOME OF THE EMBODIMENTS

The following summarizes some aspects of the present disclosure to provide a basic understanding of the discussed technology. This summary is not an extensive overview of all contemplated features of the disclosure, and is intended neither to identify key or critical elements of all aspects of the disclosure nor to delineate the scope of any or all aspects of the disclosure. Its sole purpose is to present some concepts of one or more aspects of the disclosure in summary form as a prelude to the more detailed description that is presented later.

In some embodiments of the present disclosure, methods and systems for automated intake of patient data. In some embodiments, a patient intake server may receive, at a processor and from a patient intake device, a patient intake data including unstructured patient intake data related to personal or medical information of the patient. Further, the patient intake server may select, via the processor, an engine configured to process the unstructured patient intake data. Further, the patient intake server may process, by the processor and using the engine, the unstructured patient intake data to convert the unstructured patient intake data into a structured patient intake data. In addition, the patient intake server may identify, via the processor, one or more health care providers qualified to provide the medical care to the patient based on an analysis of the structured patient intake data. In some embodiments, the patient intake server may identify an identity of the patient based on an analysis of a voice data. In some embodiments, the patient intake server may populate a structured form with data from the unstructured patient intake data to generate the structured patient intake data.

In some embodiments, a system for automated intake of patient data may include a processor and a transceiver. In some embodiments, the transceiver may be configured to receive, from a patient intake device, a patient intake data including unstructured patient intake data related to personal or medical information of the patient. In some embodiments, the processor may be configured to select an engine configured to process the unstructured patient intake data. Further, the processor may be configured to process, using the engine, the unstructured patient intake data to convert the unstructured patient intake data into a structured patient intake data. In addition, the processor may be configured to identify one or more health care providers qualified to provide the medical care to the patient based on an analysis of the structured patient intake data. In some embodiments, the processor is further configured to identify an identity of the patient based on an analysis of the voice data. In some embodiments, the processor is further configured to populate a structured form with data from the unstructured patient intake data to generate the structured patient intake data.

Some embodiments of the present disclosure disclose a non-transitory computer-readable medium (CRM) having program code recorded thereon. In some embodiments, the program code comprises code for causing a processor to receive, from a patient intake device, a patient intake data including unstructured patient intake data related to personal or medical information of the patient. The program code further comprises code for causing the processor to select an engine configured to process the unstructured patient intake data. Further, the program code comprises code for causing processor to process, using the engine, the unstructured patient intake data to convert the unstructured patient intake data into a structured patient intake data. In addition, the program code comprises code for causing the processor to identify one or more health care providers qualified to provide the medical care to the patient based on an analysis of the structured patient intake data. In some embodiments, the program code further comprises code for causing the processor to identify an identity of the patient based on an analysis of the voice data. In some embodiments, the program code further comprises code for causing the processor to populate a structured form with data from the unstructured patient intake data to generate the structured patient intake data.

In some embodiments, the unstructured patient intake data can be voice data of the patient including the personal or medical information of the patient. In some embodiments, the engine configured to process the voice data can be a voice recognition engine configured to extract the personal or medical information of the patient from the voice data. In some embodiments, the unstructured patient intake data can be image data of the patient including the personal or medical information of the patient. In some embodiments, the engine configured to process the image data can be a natural language processing (NLP) engine configured to extract the personal or medical information of the patient from the image data. In some embodiments, the engine can be an artificial intelligence (AI) neural network engine. In some embodiments, the unstructured patient intake data can be voice data of the patient including the personal or medical information of the patient.

### DETAILED DESCRIPTION

When patients need medical attention, they at first select a health care provider and then provide their personal and medical information to the provider upon arrival at the health care provider's facility. The patient data intake process, however, can be cumbersome, as most health care facilities use paper-based patient data intake systems that may be repetitive, labor intensive and likely to cause the patients to provide erroneous information. The latter may be exacerbated by the fact that the patient data intake systems may not be equipped with real-time support that can guide patients as patients provide responses to what can be confusing prompts or questions. Patient data includes personal and/or medical information of the patient such as but not limited to patient medical history, patient identifications, patient medication information, patient insurance coverage information, payment and billing information, and/or the like. As such, there is a need for systems and methods that facilitate the automated intake of patient personal and medical data while providing real-time support to the patient as needed.

Further, in some cases, it may be more efficient and beneficial to the patient if the health care provider is chosen based at least in part on the patient's personal and medical information (e.g., instead of selecting the health care provider first and providing the information later to the selected provider). Currently, and in most cases, patients select their health care providers based on the health care providers' reputation (e.g., based on ratings by rating agencies) or other reasons (e.g., convenience, etc.) that have little to do with the patients' personal and medical information. In some cases, one may wish to identify a health care provider to diagnose/treat a patient based at least in part on an analysis of the patient's personal and medical/health history, because such health care provider is more likely to have the skills, expertise and experience to provide at least satisfactory health care service to the patient. As such, there is a need for systems and methods that facilitate the identification of a health care provider for treating a patient based on the intake data of the patient (e.g., in addition to facilitating the above-noted automated intake of patient personal and medical data while providing real-time support to the patient as needed).

In some embodiments, a patient in need of medical attention may input the patient's personal and medical information into a data intake device's user interface that is powered by a patient intake engine. In some instances, the patient intake engine can be an engine executing automated software module or an artificial intelligence (AI) neural network/machine learning (ML) algorithm. The patient intake engine can be configured to receive the patient data as unstructured data, convert the unstructured data into structured data according to a predefined form for arranging patient data intake, and make predictions based on an analysis of the patient intake data (e.g., either in its structured or unstructured form). In some embodiments, the term “unstructured data” refers to data that may not be organized in a pre-defined pattern or manner, such as but not limited to free-flowing speech or voice data, text that is not arranged in a standard patient intake form (e.g., documents such as identification cards, insurance cards, letters, etc.), images, videos, etc. Further, “unstructured data” may also refer to data that may be obtained from publicly available resources (e.g., internet, third party sources, etc.), and may not also be organized in a pre-defined pattern or manner (e.g., in a pre-defined pattern or manner that may be recognized by the patient intake engine). In some embodiments, “structured data” may refer to data that is organized in a pre-defined format, for instance, data that is populated into standardized formatted fields of a patient intake form.

Artificial intelligence, implemented with neural networks and deep learning models, has demonstrated great promise as a technique for automatically analyzing real-world information with human-like accuracy. In general, such neural network and deep learning models receive input information and make predictions based on the same. Whereas other approaches to analyzing real-world information may involve hard-coded processes, statistical analysis, and/or the like, neural networks learn to make predictions gradually, by a process of trial and error, using a machine learning process. A given neural network model may be trained using a large number of training examples, proceeding iteratively until the neural network model begins to consistently make similar inferences from the training examples that a human might make. Neural network models have been shown to outperform and/or have the potential to outperform other computing techniques in a number of applications.

In some embodiments, the user interface of the patient data intake device may be in the form of a chatbot that is configured to receive audio data as input data. That is, for example, the patient may be able to speak into the user interface and the patient intake engine may process the unstructured voice data that includes information about the patient's personal, medical, health, etc., history to convert the (unstructured) voice data into data that is structured according to some predefined form. For instance, the patient may state, in conversational form, personal information such as patient name, addresses, etc., medical/health information such as medications the patient is taking or is allergic to, chronic conditions, payment information such as health insurance data, billing information, etc. In such cases, the patient intake engine may receive and process the voice data to convert the voice data into a structured data, i.e., for example, convert the voice data into data that populates the fields of a pre-defined and structured form. In some cases, the patient may state personal and medical/health information without being prompted by the chatbot, while in other cases, the chatbot may pose the questions to prompt the patient to provide the answers. In some embodiments, the audio input data may be in the form of a recorded voice data (e.g., voice recording, video, etc.) that is transmitted or provided to the patient intake engine. In such cases, the patient intake engine may extract the voice from the recorded voice data and process the voice data to convert the voice data into a structured data as discussed above.

In some embodiments, the user interface of the patient data intake device may be in the form of a chatbot that is configured to receive visual data as input data. In some cases, the visual data can be text data, image data, video data (i.e., a series of image data), etc., and the patient data intake device may be configured to receive and/or extract such visual data. For example, the patient may present the patient's identification and insurance documents to the patient intake device, which may be configured to extract from the documents at least a substantial portion of the text, image, etc., as input data (e.g., scan the documents and identify the relevant text, image, etc.). As another example, the visual data can be a video including voice and image data related to the patient's personal, health, medical, etc., information and the patient intake device may identify, and in some cases, extract the patient's personal, health, medical, etc., information as input data to be processed by the patient intake engine for conversion into structured data as discussed above. Examples of patient data intake device include smartphones, tablets, television, personal devices (e.g., portable walkie talkie type devices), smart speakers (e.g., Alexa™, google nest™, etc.) and/or the like.

In some embodiments, the intaking of the patient data may be supported or guided in real-time by a human assistant that may respond to any inquiries the patient may have. For example, the patient intake engine and/or the patient intake device may be in communication with an assistant device of an assistant that may be monitoring the patient data intake process and interceding as needed to guide the patient in inputting or providing the patient data. For example, the patient intake engine may receive a question from the patient (e.g., via the patient intake device) that the patient intake engine may not be able to or should not answer. For instance, the patient intake engine may have determined based on its training that the patient intake engine should not or could not answer certain types of questions and instead should contact the assistant device. In such cases, the patient intake engine may be configured to realize that the patient intake engine does not have an answer to the question and may send a notification to the assistant device about the patient's unanswered question. In some cases, the patient intake engine may inform or allow the patient, for example via the patient intake device's user interface, to directly contact the assistant device for help with the patient's question. For instance, the patient intake engine may activate a button on the user interface that allows the patient to directly contact the assistant device. In some cases, the assistant device may realize from monitoring the patient data intake process that the question has not been answered (e.g., after a threshold amount of time) and alert the assistant of the unanswered question without necessarily being contacted by the patient intake device.

In some embodiments, after receiving or acquiring the patient intake data (e.g., via the user interface) as input data, the patient intake device may transmit the input data to the patient intake engine for further processing to convert the received unstructured input data into data that is structured according to some pre-defined form. In some cases, the patient intake engine may include or be coupled to additional engines configured to perform such processing, and such additional engines may process the input data to generate the structured input data. For example, one of such additional engines may include a natural language processing (NLP) engine configured to analyze or parse natural language data such as text, images, etc., of the input data and convert such (unstructured) data into structured data of texts (e.g., can be populated into a structured patient intake form). For instance, the NLP engine may include an optical character recognition (OCR) software that is configured to transmit the image into text. The NLP engine may then process the converted text, which may be in unstructured form, into structured form that can be populated into a pre-defined intake form. Another example of such additional engines includes a video parsing engine configured to analyze (e.g., segment, index, etc.) the video input data to identify the patient's personal, health, medical, etc., information from the video and convert the same into the structured form; that is, for example, populate a structured patient intake form with information extracted from the video input data as a result of the analyses by the video parsing engine. In some cases, the patient intake engine may include or be coupled to a voice recognition engine configured to extract the voice of the patient from the input data and identify the identity of the patient (e.g., based on a comparison of the extracted voice to voice samples stored in a patients records database in communication with the patient intake engine).

Another example of such additional engines includes a chatbot engine configured to analyze text data, image data, video data (i.e., a series of image data), etc., input into the chatbot (e.g., of the user interface of the patient intake device) to identify/extract the patient's personal, health, medical, etc., information and convert the same into a structured form; that is, for example, populate a structured patient intake form with the extracted information. Other examples of engines that are part of, or coupled to, the patient intake engine include artificial narrow intelligence (ANI) engines, artificial super intelligence (ASI) engines, deep learning (DL) engines, machine learning (ML) engines, and/or the like. In some embodiments, the afore-mentioned various engines (e.g., patient intake engine, voice recognition engine, chatbot engine, NLP engine, video parsing engine, etc.) may be in communication with each other in converting obtained or extracted data into structured forms. For instance, the chatbot engine may obtain an image of a patient identification card, from which text may be extracted via the NLP engine, to populate a structured patient intake form with the extracted information, which results in the unstructured information in the patient identification card being converted into structured data. In some embodiments, the any one of the above-mentioned engines may include an automated software module or an artificial intelligence (AI) neural network/machine learning (ML) module to implementing the methods discussed herein for generating structured data of patients based on unstructured patient intake data.

In some embodiments, upon receiving the patient intake data and/or after processing the patient intake data to convert the data into a structured form, the patient intake engine may analyze the intake data and/or the structures intake data to identify health care providers for the patient. For example, the patient intake engine may determine based on the analyses that the patient may need a health care provider urgently and as such may select a health care provider that the patient intake engine has determined is responsive and readily available. For instance, the patient intake engine may be in communication with a health care provider database containing a list of ranked health care providers (e.g., the ranks indicating service quality, responsiveness, availability, etc. for the service type(s) the health care provider provides), and the patient intake engine may select from the health care provider database one or more health care providers to recommend to the patient based on the analyses of the received patient intake data. In some cases, the patient intake engine may process the patient intake data and generate a health care provider recommendation to the patient in real-time (e.g., while the patient is providing the patient intake data). In some cases, the patient intake engine may process the patient intake data and generate a health care provider recommendation to the patient automatically, i.e., the processing may be automated without additional input or instructions from external entities (e.g., without additional input from the assistant via the assistant device). In some cases, and in particular when requested by the patient, the assistant device may provide input or instruction to the patient intake engine to assist the patient intake engine in processing or analyzing the received patient intake data and generating a recommendation. Further, in some cases, the assistant, via the assistant device, may communicate directly with the patient to assist the patient with the patient data intake process (e.g., by answering any questions the patient may have).

In some embodiments, after receiving the patient intake data and/or after processing the patient intake data to convert the data into a structured form, the patient intake engine may configure or categorize the intake data for storage at a cloud server that is compliant with various legal regulations such as Health Insurance Portability and Accountability Act (HIPAA) that lay out strict requirements for the handling of sensitive health care data. In some cases, the stored data may be made available, in compliance with the relevant regulations, for access by the health care provider recommended or selected by the patient intake engine to diagnose/treat the patient (e.g., after approval by the patient), the patient, the assistant, or others that may be involved in provisioning health care to the patient.

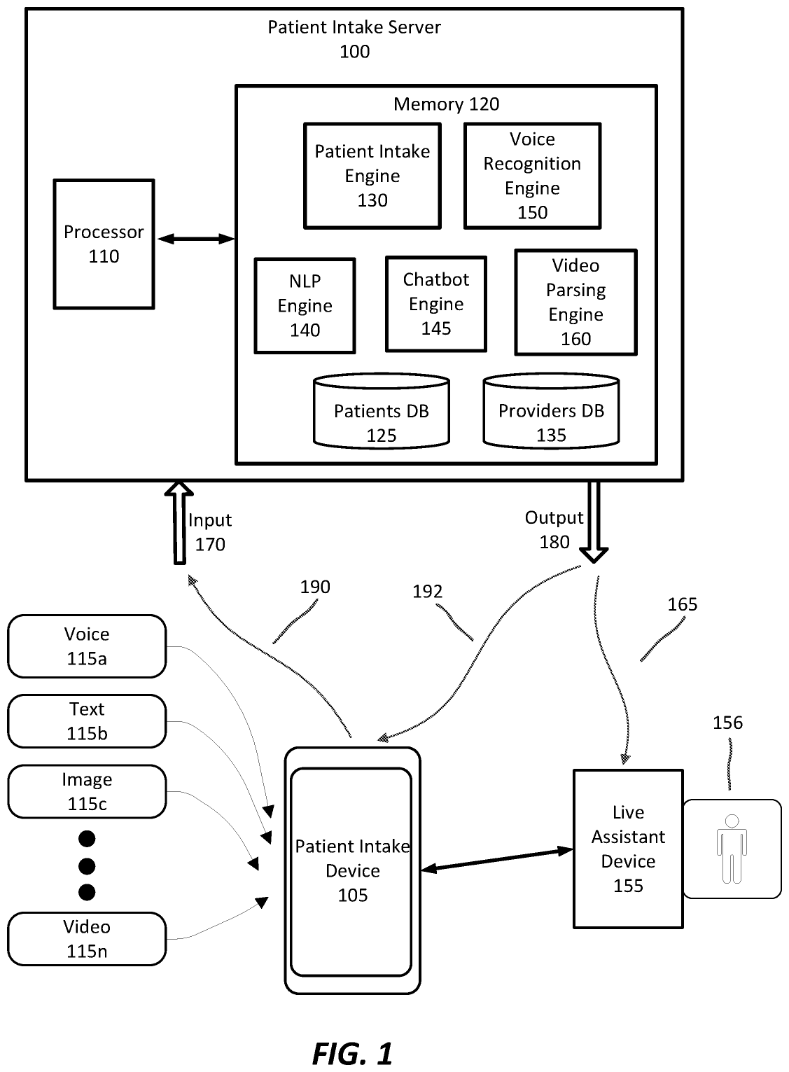

FIG. 1 is a simplified diagram of a system for automated intake of patient data, and more specifically, for generating structured database of patients and real-time identification of health care providers for the patients based on received real-time patient data, according to some embodiments. The system includes a patient intake device 105 configured to receive or obtain data related to personal and/or medical/health information of a patient. In some embodiments, the patient intake data 115 can be voice data 115a, text data 115b, image data 115c, . . . , video data 115n, etc. That is, the patient intake device 105 may have user interfaces configured to receive, extract or obtain such data. For example, the patient intake device 105 may have a recording module that is configured to record the sound, voice or speech of a patient and save or store the same as voice data 115a. As another example, the patient intake device 105 may have a camera or a scanning module (or may be coupled to a scanning module) that is configured to scan various documents to extract or obtain text and/or image data from the documents and save or store the same as text 115b, image 115c, etc. In some embodiments, the camera may be configured to capture videos of information sources (e.g., documents, the patient speaking about her/his personal and/or health/medical information) and save or store the same as video 115n. Examples of patient data intake device include smartphones, tablets, televisions, personal devices (e.g., portable walkie talkie type devices), smart speakers (e.g., Alexa™, google nest™, etc.) and/or the like, and these devices may have user interfaces configured to allow the patient to interact with, and provide the patient intake data to, the devices. In some embodiments, the different types of patient intake data 115 may be received by the patient intake device in any order.

In some embodiments, the patient intake device 105 may be configured to allow the patient to communicate with the patient intake server 100 (e.g., to the chatbot engine 145 of the patient intake server 100). For example, in some cases, the patient intake server 100 may have prompts or questions to guide the patient in providing the patient intake data 115 and the patient intake device 105 may relay these prompts or questions from the patient intake server 100 (e.g., from the chatbot engine 145 of the patient intake server 100) to the patient. For instance, the prompts or questions may be transmitted as the incoming data 192 via the afore-mentioned user interfaces of the patient intake device 105. In some embodiments, the patient may have question about the patient intake process and the patient intake device 105 may relay the patient's question to the patient intake server 100 (e.g., as the outgoing data 190).

In some embodiments, the patient intake data 115, i.e., the voice data 115a, the text data 115b, the image data 115c, . . . , the video data 115n, etc., may have already been obtained by a different device (not shown), and this device may transmit the data to the patient intake data 105 as the patient intake data 115 wirelessly or via a wired connection. For instance, the patient may have a picture (i.e., image) of their identification or insurance card on a device different from the patient intake device 105, and the different device may transmit such image to the patient intake device as image data 115c. In some embodiments, the patient may present these cards to the patient intake device 105 as part of the patient intake process and the patient intake device 105 may scan, photograph, video or otherwise obtain the information displayed on the cards and store such information as one or more of the voice data 115a, the text data 115b, the image data 115c, . . . , the video data 115n, etc. As another example, a patient may speak into the patient intake device 105 to provide the patient intake device 105 the patient's personal and/or health/medical information, and the patient intake device 105 may capture the information in one or more formats (e.g., as a text by transcribing the patient's voice, as video data by video-taping the patient's voice, as voice data by recording the sound of the voice, as image data by photographing the patient, etc.). In some embodiments, the patient intake device 105 can be in communication with or coupled to the patient intake server 100, and may transmit the received data as outgoing data 190 to the patient intake server 100, which may be received at the patient intake server 100 as an input data 170.

In some embodiments, the patient intake server 100, the patient intake device 105, and/or the live assistant device 155 may be in communication with each other via a network. For example, the patient intake device 105 may be equipped with a communications system (e.g., wireless communications system) that is in communication with a network that in turn is in communication with the patient intake server 100. In such instances, the network may allow or facilitate the patient intake device 105 to transmit the patient intake data as outgoing data 190 to be provided to the patient intake engine 100 as input 170. As another example, the network may allow or facilitate the patient intake server 100 to communicate (e.g., provide health care provider recommendations, or prompts or questions from the chatbot engine 145, etc.) to the patient intake device 105 as output 180 to be provided to the patient intake device 105 as incoming data 192.

In some cases, the network may also allow communication between the patient intake server 100 and the live assistant device 155. For example, the network may allow the human assistant 156 to communicate with the patient intake server 100 so as to monitor the communication between the patient intake device 105 and the live assistant device 155. For instance, the live assistant device 155 may receive information 165 related to the communication between the patient intake server 100 and the patient intake device 105 in real-time basis. In some instances, the network can be the Internet, representing a worldwide collection of networks and gateways to support communications between devices connected to the Internet. The network may include, for example, wired, wireless or fiber optic connections. The network can also be implemented as an intranet, a Bluetooth network, a local area network (LAN), or a wide area network (WAN). In general, the network can be any combination of connections and protocols that will support communications between computing devices, such as between the patient intake server 100, the patient intake device 105, and/or the live assistant device 155.

In some embodiments, the system for automated intake of patient data can include a live assistant device 155 in communication with or coupled to the patient intake device 105 and the patient intake server 100 (e.g., via the afore-mentioned network). As noted above, the live assistant device 155 can be configured to allow a human assistant 156 to monitor the patient intake process as a patient is providing personal and/or medical/health data to the patient intake device 105 and provide assistance to the patient, via the patient intake device 105, as needed or requested. For example, a patient may have a question during the patient data intake process which the patient intake server 100 may not be capable of answering or may not be authorized to answer (or knows that it should not answer), and in such cases, the patient intake server 100 may notify the human assistant 156 via the live assistant device 155 about the patient's questions. In some cases, the patient intake server 100 may provide the patient the opportunity to contact the live assistant device 155 to have the patient's question answered. For instance, the patient intake server 100 may activate an interactive module on the user interface of the patient intake device 105 that allows the patient to contact the live assistant device 155 (and as such the human assistant 156). For example, the interactive module can be a button or a field on the user interface of the patient intake device 105 that receives a message from the patient for transmission to the live assistant device 155.

In some embodiments, the system for automated intake of patient data can include a patient intake server 100 that includes a processor 110 coupled to memory 120. Operation of the patient intake server 100 is controlled by processor 110. And although patient intake server 100 is shown with only one processor 110, it is understood that processor 110 may be representative of one or more central processing units, multi-core processors, microprocessors, microcontrollers, digital signal processors, field programmable gate arrays (FPGAs), application specific integrated circuits (ASICs), graphics processing units (GPUs) and/or the like in patient intake server 100. Patient intake server 100 may be implemented as a stand-alone subsystem, as a board added to a computing device, and/or as a virtual machine.

Memory 120 may be used to store software executed by patient intake server 100 and/or one or more data structures used during operation of patient intake server 100. Memory 120 may include one or more types of machine readable media. Some common forms of machine readable media may include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

Processor 110 and/or memory 120 may be arranged in any suitable physical arrangement. In some embodiments, processor 110 and/or memory 120 may be implemented on a same board, in a same package (e.g., system-in-package), on a same chip (e.g., system-on-chip), and/or the like. In some embodiments, processor 110 and/or memory 120 may include distributed, virtualized, and/or containerized computing resources. Consistent with such embodiments, processor 110 and/or memory 120 may be located in one or more data centers and/or cloud computing facilities.

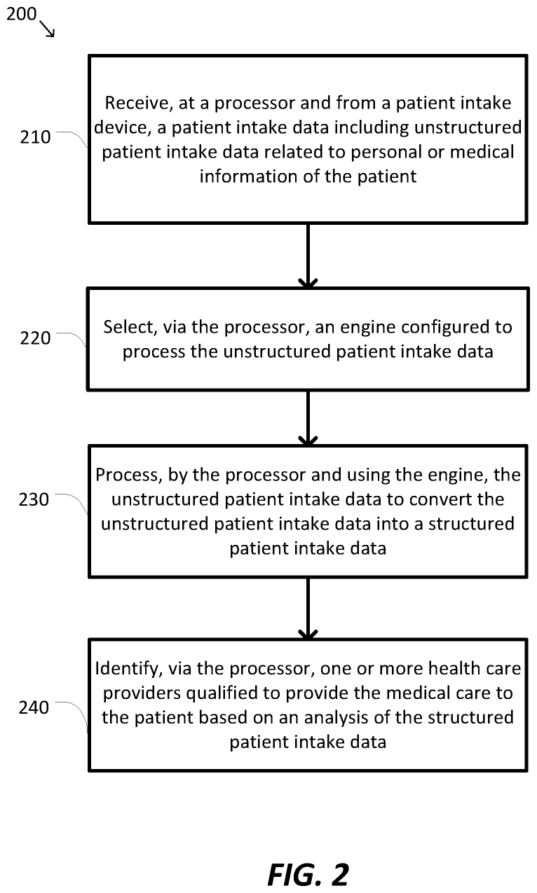

As shown, memory 120 includes a patient intake engine 130, a natural language processing (NLP) engine 140, a chatbot engine 145, a voice recognition engine 150, a video parsing engine 160, etc., that may be used to implement and/or emulate the software/neural network systems and models described further herein and/or to implement any of the methods described further herein, such as but not limited to the method described with reference to FIG. 2. One or more of the noted engines may be used, in some examples, for analyzing the input 170 that includes the patient intake data 115 to produce the output 180 that includes health care provider recommendation, a database of patients (e.g., stored in patients database (DB) 125), prompts or questions to be transmitted to the patient intake device 105, information to be provided to the live assistant device 155 to allow the human assistant to monitor the communication between the patient intake server 100 and the patient intake device 105, and/or the like.

In some examples, memory 120 may include non-transitory, tangible, machine readable media that includes executable code that when run by one or more processors (e.g., processor 110) may cause the one or more processors to perform the methods described in further detail herein. In some examples, patient intake engine 130, NLP engine 140, chatbot engine 145, voice recognition engine 150, video parsing engine 160, etc., may be implemented using hardware, software, and/or a combination of hardware and software. As shown, patient intake server 100 receives input 170, which is provided to one of more of these engines, which then may generate output 180.

In some embodiments, the memory 120 may include databases that store data received from or generated by the processor 110. In some instances, the processor 110 may generate the data by executing the patient intake engine 130, the NLP engine 140, the chatbot engine 145, the voice recognition engine 150, the video parsing engine 160, and/or the like. For example, the patients DB 125 may include data about patients generated as a result of the processing of the patient intake data 115 by the patient intake server 100 (e.g., by one or more of the afore-mentioned engines as executed by the processor 110). In some embodiments, the providers DB 135 may include data about health care providers (e.g., from which the patient intake server 100 may choose a health care provider to recommend to a patient to diagnose and/or treat the patient based on the patient intake data 115 provided by the patient). In some embodiments, the patient intake server 100 may be configured to access external databases (not shown) such as but not limited to public records about patients, health care providers, etc. For instance, the external database can be a database accessed via the internet or a database maintained by a third party. In such cases, the patient may be invited, via the patient intake device 105, to confirm the veracity of the data obtained from such databases before the data is considered or accepted as patient intake data by the patient intake server 100.

In some embodiments, as discussed above, the input 170 may include the patient intake data 115 received, extracted or obtained by the patient intake device 105 and further transmitted to the patient intake server 100. In some embodiments, the patient intake server 100 may process the received input 170 (e.g., via the engines in memory 120) to generate the output 180. That is, the output 180 can include results of analyses of the received input 140 by one or more of the patient intake engine 130, NLP engine 140, chatbot engine 145, voice recognition engine 150, video parsing engine 160, etc. For example, the received input 170 may be unstructured patient intake data, i.e., unstructured text data, unstructured video data, unstructured image data, unstructured voice data, etc., and the output 180 may include structured data text data, structured video data, structured image data, structured voice data, etc. In some embodiments, the output 180 may also include health care provider recommendations generated by the patient intake server 100 based on an analysis of the patient intake data 115 for recommending to the patient to diagnose and/or treat the patient. For instance, after a patient provides patient intake data 115 in search of a recommendation for a health care provider, the patient intake server 100 may identify a health care provider from the health care providers DB 135 based on analyses of the health intake data and recommend such a health care provider to the patient as the output 180 (e.g., to be transmitted via the outgoing data 192 and presented to the patient via the user interfaces of patient intake device 105).

As a non-limiting illustrative example, the patient intake server 100 may analyze the input (e.g., unstructured patient intake data) and/or the structured patient intake data, and the analysis may indicate that the patient has an urgent need for diagnosis/treatment. In such cases, the patient intake server 100 may use the analysis to identify, from the health care providers DB 135, one or more health care providers that are ranked or deemed to be very responsive, and transmit a list of these one or more health care providers to the patient intake device 105. For instance, the analysis may include information about the location of the patient (e.g., as extracted from the location data of the patient intake device 105), and the patient intake server 100 may use this information to identify, from the health care providers DB 135, one or more health care providers that are ranked or deemed to be very responsive and close in proximity to the patient, and transmit a list of these one or more health care providers to the patient intake device 105. As another non-limiting illustrative example, the analysis may indicate that the patient's healthcare needs may not be urgent, but may be specialized. In such cases, the patient intake server 100 may identify, based on the analysis, a specialist from the health care providers DB 135 that may be able to provide the needed diagnosis/treatment (e.g., even though the specialist may not be in close proximity to the patient or may not be available for a period of time).

In some embodiments, the output 180 may be generated on-demand, i.e., a requesting entity such as one of the patient (e.g., via the patient intake device 105), the live assistant 156 (via the live assistant device 155) or other authorized entities (e.g., health insurance providers) may request for an output 180 and the patient intake server 100 may generate the output 180 as discussed above and provide the same to a device of the requesting entity. In some cases, the patient intake server 100 may provide the output 180 automatically without any request (e.g., based on a pre-determined arrangement where a requesting entity is entitled to receive some or all of the output 180 that the patient intake server 100 generates). In some embodiments, the patient intake server 100 may provide the output 180 to the patient, the live assistant 156 or the other authorized entities, etc., in real-time as the input 170 is received by the patient intake server 100.

FIG. 2 is an example flow chart of a method 200 for automated intake of patient data. Steps of the method 200 can be executed by a computing device (e.g., a processor, processing circuit, and/or other suitable component) of a server (e.g., cloud server) or other suitable means for performing the steps. For example, the patient intake server 100 may utilize one or more components, such as the processor 110, the memory 120, the patient intake engine 130, the NLP engine 140, the chatbot engine 145, the video parsing engine 160, etc., to execute the steps of method 200. As illustrated, the method 200 includes a number of enumerated steps, but embodiments of the method 200 may include additional steps before, after, and in between the enumerated steps. In some embodiments, one or more of the enumerated steps may be omitted or performed in a different order.

At step 210, the patient intake server can receive, at a processor and from a patient intake device, a patient intake data including unstructured patient intake data related to personal or medical information of the patient. In some embodiments, a patient in need of a medical treatment may indicate to the patient intake device that the patient may be ready to provide patient intake data to the patient intake device. In some cases, the patient may not fill out a preset form such as a medical intake form. Instead, the patient may provide the patient intake data, i.e., the personal and health/medical information of the patient, in free flowing and natural form. For example, the patient may speak to the patient intake device and provide information such as but not limited to the patient's name, address, identifying numbers (e.g., social security number, identification number, phone number, etc.), health history and conditions, insurance information, etc. As discussed above, the patient intake device may be configured to capture the information the patient provides in one or more formats. That is, the patient intake device may video-tape, record the sound and/or take a photograph, etc., the patient as the patient is speaking to the patient intake device.

In some embodiments, the patient intake device 105 may be in communication with the patient intake server 100 during the patient intake process. For example, the patient intake engine 130 may be communicating with the patient intake device 105 to manage the patient intake process. In such cases, the chatbot engine 145 may be configured to generate prompts and/or questions that guide the patient in providing the patient intake data to the patient intake device 105. For example, the chatbot engine 145 may be an artificial intelligence neural network engine trained to generate questions/prompts based on input from a patient. In such cases, the chatbot engine 145 may generate prompts and/or questions for the patient based on the patient's input, and the patient intake server 100 may provide these prompts and/or questions to the patient intake device 105 during the patient data intake process. In some embodiments, in particular when the patient requests for assistance or the patient asks a question the patient intake engine cannot answer (e.g., knows not to answer based on its training), the patient intake server 100 may contact a human assistant via a live assistant device 155. In some cases, the human may be monitoring the intake process and may interject without contact from the patient intake server 100.

In some embodiment, the patient intake data received by the patient intake server may be unstructured. That is the data may not be organized in a pre-defined pattern or manner, and may be in formats such as but not limited to free-flowing speech or voice data, text not arranged in a standard patient intake form (e.g., documents such as identification cards, insurance cards, letters, etc.), images, videos, etc. In other words, the patient personal and/or health/medical information may not be contained within a structured form.

At step 220, the patient intake server may select an engine configured to process the unstructured data based on a type of the unstructured data. For example, the unstructured data may be an image that contains texts, figures, etc., and the patient intake server may select the NLP engine to process the patient intake data to extract the personal and medical/health information of the patient contained therein. As another example, the patient intake data maybe a video of the patient verbally providing personal and medical/health information and, in such cases, the patient intake server may select the video parsing engine 160 to process the video and extract the personal and medical/health information. In some embodiments, the patient intake data can be used by the patient intake server to verify the identity of the patient. For example, the patient intake server, upon receiving patient intake data that includes voice (e.g., voice recordings, video recordings containing sound of the patient, etc.) may select a voice recognition engine 150 to analyze the patient intake data to identify the patient. In such cases, the voice recognition engine (and/or the patient intake engine) may identify the patient by comparing the results of the analysis of the voice recognition engine 150 with identifying information of the patient (e.g., voice samples) stored in the patients database 125 or some other external database containing the information to which the patient intake server is coupled.

At step 230, the patient intake server may process, using the selected engine, the unstructured patient intake data to convert the unstructured patient intake data into a structured patient intake data. In some embodiments, the unstructured patient intake data may be converted into a structured form by populating a pre-determined form with the personal and/or health/medical information of the patient extracted or obtained from the unstructured patient intake data by one or more of the engines. For example, with reference to the example above, the patient intake data may be images of identification cards of a patient or voice data of a patient, and the patient intake server may have extracted the personal and/or health/medical information contained in the identification cards or the voice data with the use of one or more engines (e.g., NLP engine, video parsing engine, voice recognition engine, chatbot engine, etc.). In such cases, the patient intake server (e.g., via the patient intake engine) may populate a pre-defined intake form using the extracted information and as such may convert the unstructured patient intake data (i.e., the image data or voice data) to structured patient intake data. That is, the fields of the pre-defined intake form may be populated with the extracted information (e.g., names, addresses, identifying numbers (e.g., social security numbers, identification numbers, phone numbers, etc.), health history conditions, insurance information, etc.). It is to be noted that, in such cases, the patient may not fill out any manual or electronic form to provide personal and/or medical/health information, but rather provides such information in a natural setting (e.g., by speaking the information into a device or presenting documents that contain the information so that the information may be captured by the patient intake device). As discussed above, patient intake data provided as such may be unstructured and the patient intake server may convert such unstructured patient intake data to structured patient intake data with the use of one or more engines (e.g., AI neural network engines). In some embodiments, the patient intake server may include the structured patient intake data (e.g., the patient intake data in structure form) into a patients database (which may be part of the patient intake server 100).

At step 240, the patient intake server may identify one or more health care providers qualified to provide the medical care to the patient based on an analysis of the structured patient intake data. The health care providers may be identified based on one or more parameters, such as but not limited to the expertise of the health care provider, location or proximity of the patient to the health care provider, availability of the health care provider, service ratings of the health care provider (e.g., based on ranking system, etc.), and/or the like. In identifying the one or more health care providers, in some embodiments, the patient intake server may analyze the patient intake data (e.g., in its unstructured or structured form), information about the patient contained in the patient database of the server and/or any other patient-relevant data that may be available to the patient intake server (e.g., from a public repository or databases of external health care providers).

Computer Implemented System

In various embodiments, the methods for automated intake of patient data can be implemented via computer software or hardware. That is, as depicted in FIG. 1, the methods (e.g., 200 in FIG. 2) disclosed herein can be implemented on a computing device or server 100 that includes a processor 110, a patient intake engine 130, an NLP engine 140, a chatbot engine 145, a voice recognition engine 150, a video parsing engine 160, etc., that receive input 170 and generate output 180. In various embodiments, the computing device or server 100 can be communicatively connected to a data store or memory 120 and a display device (not shown) via a direct connection or through an internet connection.

It should be appreciated that the various engines depicted in FIG. 1 can be combined or collapsed into a single engine, component or module, depending on the requirements of the particular application or system architecture. Moreover, in various embodiments, the memory 120, the patient intake engine 130, the NLP engine 140, a chatbot engine 145, the voice recognition engine 150, the video parsing engine 160 can comprise additional engines or components as needed by the particular application or system architecture.

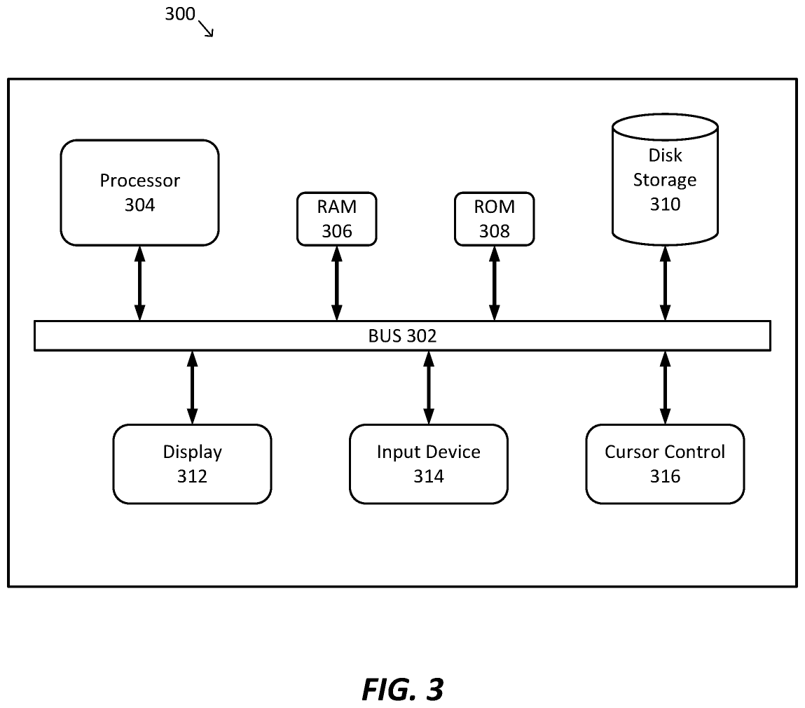

FIG. 3 is a block diagram illustrating a computer system 300 upon which embodiments of the present teachings may be implemented. In various embodiments of the present teachings, computer system 300 can include a bus 302 or other communication mechanism for communicating information and a processor 304 coupled with bus 302 for processing information. In various embodiments, computer system 300 can also include a memory, which can be a random-access memory (RAM) 306 or other dynamic storage device, coupled to bus 302 for determining instructions to be executed by processor 304. Memory can also be used for storing temporary variables or other intermediate information during execution of instructions to be executed by processor 304. In various embodiments, computer system 300 can further include a read only memory (ROM) 308 or other static storage device coupled to bus 302 for storing static information and instructions for processor 304. A storage device 310, such as a magnetic disk or optical disk, can be provided and coupled to bus 302 for storing information and instructions.

In various embodiments, computer system 300 can be coupled via bus 302 to a display 312, such as a cathode ray tube (CRT) or liquid crystal display (LCD), for displaying information to a computer user. An input device 314, including alphanumeric and other keys, can be coupled to bus 302 for communication of information and command selections to processor 304. Another type of user input device is a cursor control 316, such as a mouse, a trackball or cursor direction keys for communicating direction information and command selections to processor 304 and for controlling cursor movement on display 312. This input device 314 typically has two degrees of freedom in two axes, a first axis (i.e., x) and a second axis (i.e., y), that allows the device to specify positions in a plane. However, it should be understood that input devices 314 allowing for 3-dimensional (x, y and z) cursor movement are also contemplated herein.

Consistent with certain implementations of the present teachings, results can be provided by computer system 300 in response to processor 304 executing one or more sequences of one or more instructions contained in memory 306. Such instructions can be read into memory 306 from another computer-readable medium or computer-readable storage medium, such as storage device 310. Execution of the sequences of instructions contained in memory 306 can cause processor 304 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 304 for execution. Such a medium can take many forms, including but not limited to, non-volatile media, volatile media, and transmission media. Examples of non-volatile media can include, but are not limited to, dynamic memory, such as memory 306. Examples of transmission media can include, but are not limited to, coaxial cables, copper wire, and fiber optics, including the wires that comprise bus 302.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 304 of computer system 300 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 300 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 300, whereby processor 304 would execute the analyses and determinations provided by these engines, subject to instructions provided by any one of, or a combination of, memory components 306/308/310 and user input provided via input device 314.

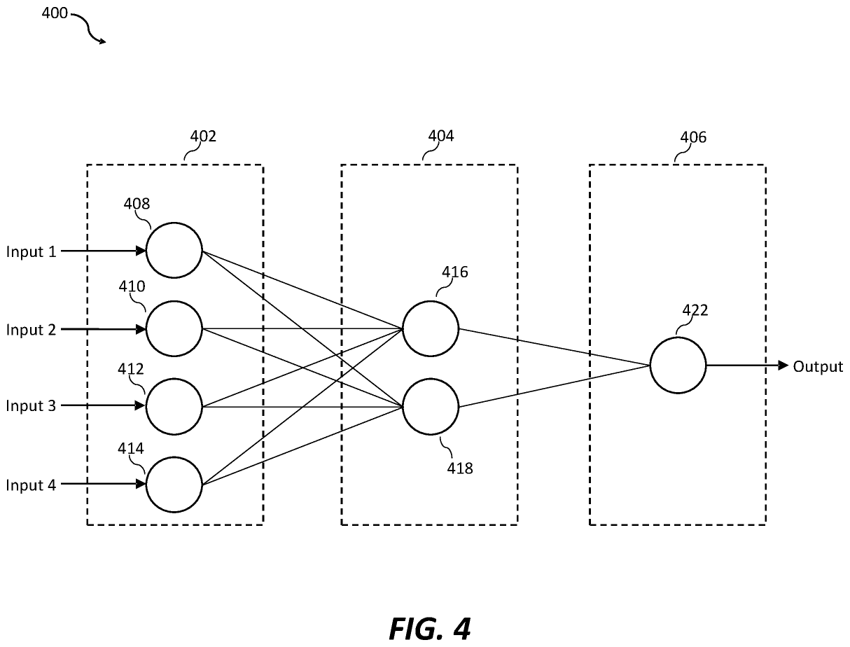

FIG. 4 illustrates an example neural network that can be used to implement a computer-based model according to various embodiments of the present disclosure. In various embodiments, the methods for automated intake of patient data can be implemented via a machine learning/neural network module. That is, as depicted in FIG. 1, the methods (e.g., 200 in FIG. 2) disclosed herein can be implemented on a computing device or server 100 that includes a patient intake engine 130, an NLP engine 140, a chatbot engine 145, a voice recognition engine 150, a video parsing engine 160, etc., that may each include a neural network or a machine learning algorithm for executing or implementing the methods for automated intake of patient data discussed herein. FIG. 4 illustrates an example neural network that can be used to implement a computer-based model or engine according to various embodiments of the present disclosure. As shown, the artificial neural network 400 includes three layers—an input layer 402, a hidden layer 404, and an output layer 406. Each of the layers 402, 404, and 406 may include one or more nodes. For example, the input layer 402 includes nodes 408-414, the hidden layer 404 includes nodes 416-418, and the output layer 406 includes a node 422. In this example, each node in a layer is connected to every node in an adjacent layer. For example, the node 408 in the input layer 402 is connected to both of the nodes 416, 418 in the hidden layer 404. Similarly, the node 416 in the hidden layer is connected to all of the nodes 408-414 in the input layer 402 and the node 422 in the output layer 406. Although only one hidden layer is shown for the artificial neural network 400, it has been contemplated that the artificial neural network 400 used to implement the machine learning algorithms of the neural networks included in the patient intake engine 130, the NLP engine 140, the chatbot engine 145, the voice recognition engine 150, the video parsing engine 160, etc., may include as many hidden layers as necessary or desired.

In this example, the artificial neural network 400 receives a set of input values and produces an output value. Each node in the input layer 402 may correspond to a distinct input value (e.g., different features of the unstructured patient intake data). In some embodiments, each of the nodes 416-418 in the hidden layer 404 generates a representation, which may include a mathematical computation (or algorithm) that produces a value based on the input values received from the nodes 408-414. The mathematical computation may include assigning different weights to each of the data values received from the nodes 408-414. The nodes 416 and 418 may include different algorithms and/or different weights assigned to the data variables from the nodes 408-414 such that each of the nodes 416-418 may produce a different value based on the same input values received from the nodes 408-414. In some embodiments, the weights that are initially assigned to the features (or input values) for each of the nodes 416-418 may be randomly generated (e.g., using a computer randomizer). The values generated by the nodes 416 and 418 may be used by the node 422 in the output layer 406 to produce an output value for the artificial neural network 400. When the artificial neural network 400 is used to implement the machine learning algorithms of the neural networks included in the patient intake engine 130, the NLP engine 140, the chatbot engine 145, the voice recognition engine 150, the video parsing engine 160, etc., the output value produced by the artificial neural network 400 may include structured patient intake data.

The artificial neural network 400 may be trained by using training data. For example, the training data herein may be unstructured patient intake data discussed above (e.g., unstructured text, image, video, audio, etc., patient data). By providing training data to the artificial neural network 400, the nodes 416-418 in the hidden layer 404 may be trained (adjusted) such that an optimal output is produced in the output layer 406 based on the training data. By continuously providing different sets of training data, and penalizing the artificial neural network 400 when the output of the artificial neural network 400 is incorrect (e.g., when incorrectly identifying or failing to identify unstructured data that can be converted into structured data), the artificial neural network 400 (and specifically, the representations of the nodes in the hidden layer 404) may be trained (adjusted) to improve its performance in data classification. Adjusting the artificial neural network 400 may include adjusting the weights associated with each node in the hidden layer 404.

Although the above discussions pertain to an artificial neural network as an example of machine learning, it is understood that other types of machine learning methods may also be suitable to implement the various aspects of the present disclosure. For example, support vector machines

(SVMs) may be used to implement machine learning. SVMs are a set of related supervised learning methods used for classification and regression. A SVM training algorithm—which may be a non-probabilistic binary linear classifier—may build a model that predicts whether a new example falls into one category or another. As another example, Bayesian networks may be used to implement machine learning. A Bayesian network is an acyclic probabilistic graphical model that represents a set of random variables and their conditional independence with a directed acyclic graph (DAG). The Bayesian network could present the probabilistic relationship between one variable and another variable. Another example is a machine learning engine that employs a decision tree learning model to conduct the machine learning process. In some instances, decision tree learning models may include classification tree models, as well as regression tree models. In some embodiments, the machine learning engine employs a Gradient Boosting Machine (GBM) model (e.g., XGBoost) as a regression tree model. Other machine learning techniques may be used to implement the machine learning engine, for example via Random Forest or Deep Neural Networks. Other types of machine learning algorithms are not discussed in detail herein for reasons of simplicity and it is understood that the present disclosure is not limited to a particular type of machine learning.

Recitations of some Embodiments of the Present Disclosure

Embodiment 1: A method, comprising: receiving, at a processor and from a patient intake device, a patient intake data including unstructured patient intake data related to personal or medical information of the patient; selecting, via the processor, an engine configured to process the unstructured patient intake data; processing, by the processor and using the engine, the unstructured patient intake data to convert the unstructured patient intake data into a structured patient intake data; and identifying, via the processor, one or more health care providers qualified to provide the medical care to the patient based on an analysis of the structured patient intake data.

Embodiment 2: The method of embodiment 1, wherein the unstructured patient intake data is voice data of the patient including the personal or medical information of the patient.

Embodiment 3: The method of embodiment 2, wherein the engine configured to process the voice data is a voice recognition engine configured to extract the personal or medical information of the patient from the voice data.

Embodiment 4: The method of embodiment 2 or 3, further comprising identifying an identity of the patient based on an analysis of the voice data.

Embodiment 5: The method of any of embodiments 1-4, wherein the unstructured patient intake data is image data of the patient including the personal or medical information of the patient.

Embodiment 6: The method of embodiment 5, wherein the engine configured to process the image data is a natural language processing (NLP) engine configured to extract the personal or medical information of the patient from the image data.

Embodiment 7: The method of any of embodiments 1-6, further comprising populating a structured form with data from the unstructured patient intake data to generate the structured patient intake data.

Embodiment 8: The method of any of embodiments 1-7, wherein the engine is an artificial intelligence (AI) neural network engine.

Embodiment 9: A system, comprising: a processor and a transceiver coupled to the processor, the system configured to perform the methods of embodiments 1-8.

Embodiment 10: A non-transitory machine-readable medium having stored thereon machine-readable instructions executable to cause a machine to perform the methods of embodiments 1-8.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and in a manner consistent with the scope of the embodiments disclosed herein.

What is claimed is:

1. A method, comprising:
receiving, at a processor and from a patient intake device, a patient intake data including unstructured patient intake data related to personal or medical information of the patient;
selecting, via the processor, an engine configured to process the unstructured patient intake data;
processing, by the processor and using the engine, the unstructured patient intake data to convert the unstructured patient intake data into a structured patient intake data; and
identifying, via the processor, one or more health care providers qualified to provide the medical care to the patient based on an analysis of the structured patient intake data.

2. The method of claim 1, wherein the unstructured patient intake data is voice data of the patient including the personal or medical information of the patient.

3. The method of claim 2, wherein the engine configured to process the voice data is a voice recognition engine configured to extract the personal or medical information of the patient from the voice data.

4. The method of claim 2, further comprising identifying an identity of the patient based on an analysis of the voice data.

5. The method of claim 1, wherein the unstructured patient intake data is image data of the patient including the personal or medical information of the patient.

6. The method of claim 5, wherein the engine configured to process the image data is a natural language processing (NLP) engine configured to extract the personal or medical information of the patient from the image data.

7. The method of claim 1, further comprising populating a structured form with data from the unstructured patient intake data to generate the structured patient intake data.

8. The method of claim 1, wherein the engine is an artificial intelligence (AI) neural network engine.

9. A system, comprising:
a transceiver configured to:
receive, from a patient intake device, a patient intake data including unstructured patient intake data related to personal or medical information of the patient; and
a processor configured to:
select an engine configured to process the unstructured patient intake data;
process, using the engine, the unstructured patient intake data to convert the unstructured patient intake data into a structured patient intake data; and
identify one or more health care providers qualified to provide the medical care to the patient based on an analysis of the structured patient intake data.

10. The system of claim 9, wherein the unstructured patient intake data is voice data of the patient including the personal or medical information of the patient.

11. The system of claim 10, wherein the engine configured to process the voice data is a voice recognition engine configured to extract the personal or medical information of the patient from the voice data.

12. The system of claim 10, wherein the processor is further configured to identify an identity of the patient based on an analysis of the voice data.

13. The system of claim 9, wherein the unstructured patient intake data is image data of the patient including the personal or medical information of the patient.

14. The system of claim 13, wherein the engine configured to process the image data is a natural language processing (NLP) engine configured to extract the personal or medical information of the patient from the image data.

15. The system of claim 9, wherein the processor is further configured to populate a structured form with data from the unstructured patient intake data to generate the structured patient intake data.

16. A non-transitory computer-readable medium (CRM) having program code recorded thereon, the program code comprising:
code for causing a processor to receive, from a patient intake device, a patient intake data including unstructured patient intake data related to personal or medical information of the patient;
code for causing the processor to select an engine configured to process the unstructured patient intake data;
code for causing processor to process, using the engine, the unstructured patient intake data to convert the unstructured patient intake data into a structured patient intake data; and
code for causing the processor to identify one or more health care providers qualified to provide the medical care to the patient based on an analysis of the structured patient intake data.

17. The non-transitory CRM of claim 16, wherein the unstructured patient intake data is voice data of the patient including the personal or medical information of the patient.

18. The non-transitory CRM of claim 16, wherein the unstructured patient intake data is image data of the patient including the personal or medical information of the patient.

19. The non-transitory CRM claim 18, wherein the engine configured to process the image data is a natural language processing (NLP) engine configured to extract the personal or medical information of the patient from the image data.

20. The non-transitory CRM of claim 16, wherein the program code further comprises code for causing the processor to populate a structured form with data from the unstructured patient intake data to generate the structured patient intake data.
